(19) United States
(12) Patent Application Publication
(10) Pub. No.: US 2017/0156854 A1
(43) Pub. Date: Jun. 8, 2017

(54) CURVED FIBER ARRANGEMENT FOR PROSTHETIC HEART VALVES

(71) Applicant: Children's Medical Center Corporation, Boston, MA (US)

(72) Inventor: Peter Hammer, Needham, MA (US)

(73) Assignee: Children's Medical Center Corporation, Boston, MA (US)

(21) Appl. No.: 15/208,343

(22) Filed: Jul. 12, 2016

Related U.S. Application Data
(63) Continuation of application No. 13/996,567, filed on Jun. 21, 2013, now abandoned.
(60) Provisional application No. 61/427,930, filed on Dec. 29, 2010.

(51) Int. Cl. A61F 2/24

(52) U.S. Cl. CPC A61F 2/24; A61F 2/2412; A61F 2/2439; A61F 2002/068

(57) ABSTRACT

A leaflet including fibers oriented at an angle relative to at least one free edge of the leaflet. A leaflet including mechanisms for increasing coaptation height, preventing billowing, and reducing stress in critical regions of the leaflet. A prosthetic heart valve, including three leaflets operatively attached together. A method of using a prosthetic heart valve, by applying pressure to the valve, forming a pocket with material of three leaflets operatively attached together and increasing coaptation height, reducing billowing of the leaflets toward a ventricle, and reducing stress in critical regions of the leaflet. A chorded valve including at least one leaflet, wherein bundles of fibers exit said free edges as tethers an can be anchored to tissue. A method of using the chorded valve, by anchoring the tethers to tissue, forming a pocket with the material of leaflets and increasing coaptation height, and reducing billowing of leaflets toward an atrium.

### RELATED APPLICATIONS

This application is a continuation of and claims priority under 35 U.S.C. §120 to U.S. patent application Ser. No. 13/996,567, filed Jun. 21, 2013 and entitled “CURVED FIBER ARRANGEMENT FOR PROSTHETIC HEART VALVES,” which is a national stage filing under 35 U.S.C. §371 of International Application No. PCT/US2011/067745, filed Dec. 29, 2011 and entitled “CURVED FIBER ARRANGEMENT FOR PROSTHETIC HEART VALVES,” which claims priority under 35 U.S.C. §119(e) to U.S. Provisional Application No. 61/427,930, filed Dec. 29, 2010, each of which is incorporated herein by reference in its entirety.

### GRANT INFORMATION

Research in this application was supported in part by a grant from the National Institute of Health (NIH Grant No. R01-HL73647). The Government has certain rights in the invention.

### BACKGROUND OF THE INVENTION

1. Technical Field

The present invention relates to heart valves. In particular, the present invention relates to heart valves that have a curved or bent fiber arrangement that can be used to control the 3-dimensional shape of a pressurized membrane.

2. Background Art

Artificial heart valves have been known for years and have been used to replace native valves that have become faulty through disease. The artificial heart valves themselves should ideally be designed to last for the life of the patient, in many cases in excess of thirty-five years, equivalent to over 1.8 billion heartbeats. Heart valves that can be replaced include aortic and pulmonary valves, as well as mitral and tricuspid valves.

As to the operation of normal heart valves, they open and close largely passively in response to changes in pressure in the heart chambers or great vessels i.e. aorta and pulmonary artery, which they connect. For example, the aortic valve situated between the left ventricle and the ascending aorta, opens when the rising pressure in the contracting left ventricle exceeds that in the aorta. Blood in the ventricle is then discharged into the aorta. The valve closes when the pressure in the aorta exceeds that in the ventricle.

Problems occur with the native valves when they fail to function properly through disease or trauma. Faulty valves exhibit leakage in the closed position, i.e. regurgitation, obstruction to flow in the open position, i.e. stenosis, or a combination of the two, i.e. mixed valve disease. The response of the heart to faulty valves is demonstrated by changes in the left ventricle which ensue in response to malfunction of the aortic valve. Initially the heart compensates by an increase in muscle mass i.e. hypertrophy, a process that is to some extent reversible. Eventually, however, the heart can compensate no longer and begins to dilate. This latter process is irreversible even with replacement of the faulty valve. Untreated, it leads to end stage heart failure and ultimately death. Valve replacement has become a routine operation in the developed world for patients shown to have heart valve disease who have not yet reached the stage of irreversible, end stage heart failure.

In the past, there have been two broad types of valves that have been used in replacement procedures: mechanical valves and biological valves.

Mechanical valves are constructed from rigid materials. The design of these valves takes one of three general forms: ball and cage, tilting disk or bileaflet prostheses. In general, mechanical valves have in their favor long term durability intrinsic to the very tough materials from which they are made. With a few notable exceptions, such as the well publicized Shiley CC series, mechanical failure of these valves has been very rare. Followup for some of the first generation ball and cage valves now exceeds thirty years and the longevity of more recent designs such as the latest bileaflet prostheses is expected to match these results.

The principal shortcomings of mechanical valves, however, are the need for long term anticoagulation, the tendency to cause red blood cell haemolysis in some patients and the noise created by repeated opening and closing of the valve which patients find very disturbing. Anticoagulation requires the patient to take a regular daily dose of medication that prolongs the clotting time of blood. The exact dose of medication, however, needs to be tailored to the individual patient and monitored regularly through blood tests. Apart from the inconvenience and potential for non-compliance imposed by this regimen, inadvertent over-coagulation or under-coagulation is not uncommon. Under-coagulation can lead to thrombosis of the valve itself or embolism of clotted blood into the peripheral circulation where it can cause a stroke or local ischaemia, both potentially life threatening conditions. On the other hand, overcoagulation can cause fatal spontaneous haemorrhage. It is clear therefore that anticoagulation, even in the most expert hands, is associated with finite risks of morbidity and mortality. This risk accrues significantly over the patient's lifetime. For this reason, some surgeons avoid the use of mechanical prostheses, where possible.

Hemolysis is the lysis of red blood cells in response to stresses imposed on those cells as blood crosses mechanical valves. Significant hemolysis causes anemia. These patients are required to have regular replacement blood transfusions with the attendant inconvenience, expense, and risks which that entails.

Haemolysis and the need for anticoagulation result principally from microcavitation and regional zones of very high shear stress created in the flow of blood through mechanical valves. These physical phenomena are imposed on elements in the blood, i.e. red blood cells and platelets, responsible for activating the clotting cascade occasioned by the design of existing prostheses having either a rigid ball and cage, a rigid disk or two rigid leaflets.

Finally, mechanical valves may not be suitable for small patients as a significant gradient exists across these valves in the smaller sizes.

Biological valves are constructed from a variety of naturally occurring tissues taken from animals and fixed by treatment with glutaraldehyde or similar agent. Materials that have been used include dura mater from the lining of the brain, pericardium from the sac enclosing the heart or valve tissue itself from pigs and cows. These materials are used to fashion replacement heart valve leaflets and in the past have been assembled with the aid of a rigid supporting frame or stent. More recently leaflets made from these materials have been supported without the aid of a rigid frame and are fixed over flexible materials such as Dacron. The latter are referred to as stentless valves.

In contradistinction to mechanical valves, biological valves have flow hemodynamics that resemble the flow through native heart valves. In general, they do not therefore require lifelong anticoagulation and do not cause red cell hemolysis. Furthermore, very little residual gradient can be measured across even the smallest available stentless biological valves. Additionally, biological valves function inaudibly.

Unfortunately, however, biological valves suffer from degenerative changes over time. At least 50% of porcine valves implanted in the aortic position fail within 10-15 years post operatively. Furthermore, this risk is amplified in the mitral position and in younger patients where failure of porcine aortic valves is almost universal by five years. Progressive deterioration of biological valves manifests itself either as obstruction to forward flow through the valve in the open position, i.e. stenosis, or more commonly as tears in the valve leaflets that cause leakage in the closed position, i.e. regurgitation.

To summarize, the configuration of biological valves allows them to function inaudibly without the risks of thrombosis or hemolysis. However, the biological materials from which they are made do not have the durability to last the patient's potential lifetime.

A valve that combines the durability of man-made materials with the hemodynamics of a biological valve would be inaudible, free from the problems of anticoagulation and risk of hemolysis and yet exhibit the necessary durability to last the patient's lifetime.

Several valves of this type have been described in the prior art. For example, U.S. Pat. No. 6,726,715 to Sutherland discloses valve leaflets that have strands, fibers, or yarns aligned along stress lines so that reinforcement of leaflet occurs. The fiber direction is parallel to the free edge of the leaflet, resulting in a leaflet that is relatively stiff in the direction parallel to the leaflet free edge and relatively compliant in the perpendicular (cross-fiber) direction (see FIGS. 12 and 13 of Sutherland). Such an arrangement of fibers does not result in optimal performance of the leaflet. Therefore, there is a need for a man-made valve that overcomes these problems.

### SUMMARY OF THE INVENTION

The present invention provides for a leaflet including fibers oriented at an angle relative to at least one free edge of the leaflet.

The present invention provides for a leaflet including a mechanism for increasing coaptation height, preventing billowing, and reducing stresses in critical regions of the leaflet.

The present invention further provides for a prosthetic heart valve, including three leaflets operatively attached together.

The present invention also provides for a method of using a prosthetic heart valve by applying pressure to the valve, forming a pocket with material of three leaflets operatively attached together and increasing coaptation height, reducing billowing of the leaflets toward a ventricle, and reducing stresses in critical regions of the leaflet.

The present invention provides for a chorded valve comprising at least one leaflet including bent or curved fibers with respect to at least one free edge of the leaflet, wherein bundles of fibers exit the free edges as tethers and can be anchored to tissue.

The present invention also provides for a method of using the chorded valve by anchoring the tethers to tissue, forming a pocket with the material of leaflets and increasing coaptation height, reducing billowing of leaflets toward an atrium, and reducing stresses in critical regions of the leaflet.

### BRIEF DESCRIPTION ON THE DRAWINGS

Other advantages of the present invention will be readily appreciated as the same becomes better understood by reference to the following detailed description when considered in connection with the accompanying drawings, wherein:

FIG. 1A is a drawing showing fibers that are oriented parallel to the leaflet free edge in prosthetic valves of the prior art, FIG. 1B is a drawing showing the fibers of the leaflet of the present invention forming v-shaped patterns (or alternatively, smooth arcs) across the leaflet, and FIG. 1C is a three-dimensional view of three leaflets combined to form a tri-leaflet valve and pressure is applied forcing the leaflets to close;

FIG. 2 is a sketch showing a side view of a pressurized leaflet (solid gray curves represents the leaflet profile for the case where fibers are oriented parallel to the free edge, and solid black curves represent the case of v-shaped (bipennate) or curved fibers;

FIG. 3 is a drawing of a mitral type valve incorporating v-shaped fibers within the leaflet (shown in gray) that continue outside the leaflet emulating the chordae tendineae that tether the native mitral valve leaflets;

FIGS. 4A-4C are drawings showing straight fibers, curved-uniform fibers, and curved-nonuniform fibers tested in the example;

FIG. 5A is a drawing showing fiber direction in a single aortic valve leaflet, and FIGS. 5B-5C are drawings showing pressure loading to a valve;

FIGS. 6A-6B are models of straight fiber valves upon pressure loading, FIGS. 6C-6D are models of curved-uniform fiber valves upon pressure loading, and

FIG. 6E is a comparison of the seal and billow of the straight fibers versus the curved-uniform fibers;

FIG. 7A is a graph of leaflet stress in a direction perpendicular to the fibers in the curved-uniform leaflet, and FIG. 7B is a graph of leaflet stress in the curved-nonuniform leaflet;

FIG. 8A is a summary of the mechanism of membrane deformation, FIG. 8B is a drawing of the membrane indicating the direction of deformation of the membrane, and FIG. 8C is a drawing of deformation of the membrane when pressurized, and

FIG. 9 is a schematic representation of a leaflet attached to a flexible conduit.

### DETAILED DESCRIPTION

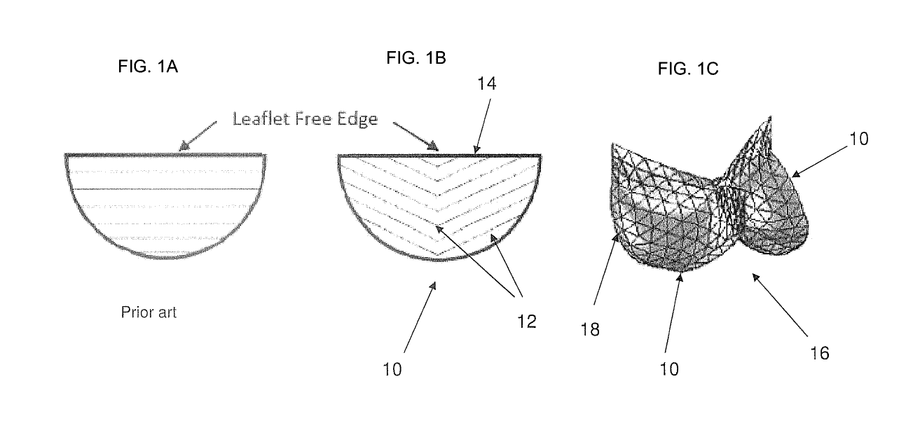

The present invention provides for a leaflet 10 including bent or curved fibers 12 with respect to at least one free edge 14 of the leaflet 10, shown generally in FIG. 1B. In other words, the fibers 12 are not parallel to the free edge 14, as shown in FIG. 1A, but are oriented at an angle relative to the free edge 14.

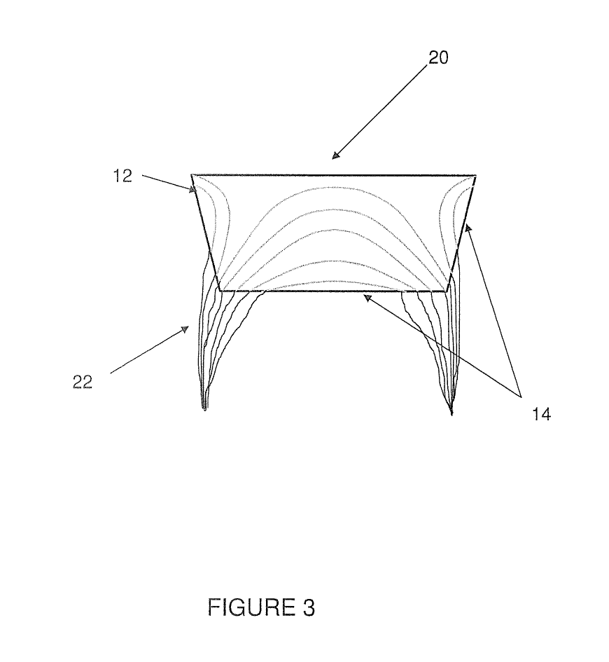

More specifically, the fibers 12 can be arranged in a V shape that opens toward a single free edge 14 as shown in FIG. 1B. The leaflet 10 can have a single free edge 14 or multiple free edges 14. The fibers 12 can also be arranged to open toward at least two free edges 14 such as those shown in FIG. 3. In other words, the fibers 12 can be arranged in a uniform or non-uniform manner throughout the leaflet 10. Such an arrangement is useful when the leaflet must form a seal along all of its free edges 14, as further discussed below. Having the fibers 12 be orientated at an angle relative to each free edge 14 can increase coaptation height and tension in the leaflet 10 to prevent billowing.

The design of the leaflet 10 is based on the fact that reinforcing fibers in a planar membrane can be arranged or oriented to achieve specific three-dimensional features in the membrane when it is loaded by pressure. Rather than orienting fibers in straight lines across a membrane, as has been done in prior art leaflets, the fibers 12 are orientated to form bent or curved paths. The leaflet 10 has a pressurized conformation and a non-pressurized conformation. When pressure is applied to the membrane surface, membrane tension tends to straighten the bent or curved fibers, causing displacement of portions of the membrane in directions tangent to the membrane surface. The more compliant the membrane relative to the compliance of the reinforcing fibers, the larger the magnitude of these tangent displacements of the membrane.

The leaflet 10 is generally made from one or more sheets of plastic materials such as TEFLON® (DuPont) (polytetrafluoroethylene), polyurethane, MYLAR® (DuPont) (biaxially-oriented polyethylene terephthalate), or other types of laminatable material. The fibers 12 can be carbon fibers, polyester fibers such as VECTRAN® (Hoescht Celanse), fibers made from the aramids KEVLAR® (DuPont), TWARON® (Akzo), TECHNORA® (Teijin), and also polyethylene fibers such as Dynema (DSM), CERTRAN® (Hoescht Celanese), or SPECTRA® (Allied-Signal Corporation). By current practices, leaflets are cut from the biological material so that the fiber direction is parallel to the free edge of the leaflet (as shown in FIG. 1A), resulting in a leaflet that is relatively stiff in the direction parallel to the leaflet free edge and relatively compliant in the perpendicular (cross-fiber) direction. In contradistinction, the leaflet of the present invention is formed by cutting one half of a leaflet obliquely with respect to the fiber 12 direction in the material, and cutting a second half similarly to form a mirror image of the first, as shown in FIG. 1B. For a tissue-engineered valve, a scaffold of fibers based on the fiber arrangement disclosed herein can be manufactured (e.g. by weaving or electrospinning). For a bioprosthetic valve, half of the leaflet can be cut obliquely from pericardium (which has roughly parallel fiber structure), another half can be cut as a mirror image, and then the two halves can be sewn up the midline. For a valve with leaflets made from polymers, the fibers can be cast into an elastic matrix in bent/curved form, or they can be sandwiched and bonded between two layers of the elastic matrix. Any other appropriate methods known in the art can also be used in creating the leaflet 10.

Preferably, the leaflet 10 is used as in prosthetic heart valve 16 including three leaflets 10 attached to a frame 18. A common design for heart valves consists of three leaflets attached to a frame, where the leaflets are made of biological materials that have a preferential fiber direction. While all three of the leaflets can be the leaflet 10 of the present invention, either one or two leaflets 10 can also be used with other types of leaflets to create the valve 16. When the new leaflet 10 of the present invention, exhibiting v-shaped or “bipennate” fiber orientation when the leaflet 10 is in the unstressed state, is arranged with two more such leaflets 10 into a tri-leaflet valve 16 and pressurized, it now undergoes deformations and displacements tangent to the leaflet surface and that improves the ability of the closed valve to prevent regurgitation (backflow). Three leaflets 10 can also be used in a stentless valve without the frame 18 that is attached to a flexible conduit or sewn directly into the wall of the outflow vessel.

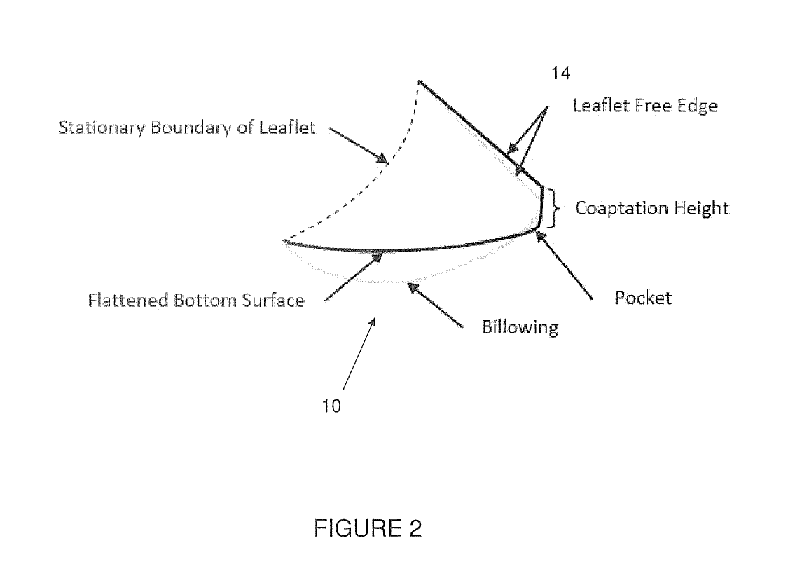

There are two different conformal changes caused by the novel fiber arrangement of the leaflet 10. First, material is pushed along the leaflet midline toward the free edge of the leaflet. However, the midpoint of the free edge is not subject to this force due to fiber straightening, so excess leaflet material accumulates along the distal portion of the leaflet midline, forming a “pocket”. This pocket greatly increases the amount of overlap of the three leaflets at the center of the valve 16 (FIG. 2). This overlap, referred to as coaptation height by cardiac surgeons, is an important feature of tri-leaflet valves, with larger coaptation heights corresponding to more robust valve function. Second, increased tension on the proximal portion of the leaflet midline, which flattens the surface of the closed valve, results in less billowing of the leaflets 10 toward the ventricle (FIG. 2). Billowing is detrimental to heart function because it both reduces cardiac filling and dissipates energy in the pressurized outflow vessel.

Another important consequence of the novel fiber arrangement in the leaflet 10 is a decrease in peak stress in the fibers 12 as pressure is applied to the valve 16, i.e. stress is reduced in critical areas of the leaflet 10. This is due to the fact that the straightening of the fibers 12 with application of pressure is opposed by the elastic deformation of the leaflet 10 in the direction of the leaflet midline. The result is that the sudden rise in transvalvular pressure causes a gradual increase in tension in the fibers 12 as the leaflet 10 stretches along its midline. This is in contrast to the sudden, impulsive jump in tension that occurs in fibers 12 that run parallel to the leaflet free edge 14. This decrease in peak fiber tension with each loading cycle of the valve 16 significantly increases its durability.

Therefore, the present invention includes a method of using the prosthetic heart valve 16, by forming a pocket with the material of the leaflets 10 and increasing coaptation height, reducing billowing of leaflets toward a ventricle, and reducing stresses in critical regions of the leaflet 10.

This mechanism is able to redistribute leaflet material to where it is needed near the center of the closed valve using strictly passive means (i.e., actuated by aortic pressure, not through a metabolically active mechanism like muscle contraction). When the valve 16 opens to allow ejection of blood from the ventricle, transleaflet pressure vanishes, allowing the leaflet 10 to resume its unstressed state with v-shaped or curved fibers 12. Designing a valve with this mechanism, it is possible to develop a valve with adequate coaptation that has a smaller leaflet midline length in the absence of membrane tension, i.e., when the valve is open and blood is flowing through. This has the advantages of reduced outflow resistance and less material used for the valve. The latter has implications for stented valves, which are deployed by catheter where there are limits to the total amount of material that can be fit into a valve. Another advantage that this novel fiber arrangement confers upon the closed valve 16 is the decreased tension in the free edge 14 (i.e., shorter free edge length, FIG. 2). This allows prosthetic valve leaflets to be made from thinner materials, which, again, is important for stented valves designed for catheter deployment.

In addition to tri-leaflet replacement valves (which mimic the design of the native aortic and pulmonary valves), the fiber arrangement scheme described above can also be applied to prosthetic valves or replacement leaflets for chorded valves 20, i.e., those mimicking the mitral and tricuspid valves. The chorded valves 20 can be formed from least one leaflet 10, as well as multiple leaflets 10. Again, the v-shaped fibers 12 of the leaflet 10 are arranged to “open” toward the free edge 14 (FIG. 3). An important difference in this case is that bundles of leaflet fibers 12 exit the leaflet free edges 14 as tethers 22 and can be anchored to papillary muscles in the apex of the ventricle. However the role of the v-shaped (or curved) fibers 12 is the same: they force the leaflet 10 toward the coaptation region, prevent the leaflet 10 from billowing toward the atrium, and form a pocket in the leaflet 10 adjacent to the line of coaptation.

Therefore, the present invention also includes a method of using a chorded valve, by anchoring the tethers to tissue, forming a pocket with the material of the leaflets and increasing coaptation height, reducing billowing of leaflets toward an atrium, and reducing stress in critical regions of the leaflet.

The invention is further described in detail by reference to the following experimental examples. These examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

Aortic valve leaflets are known to exhibit anisotropic mechanical response due to collagen fibers running in a preferred direction. Prosthetic valves and leaflet grafts for valve repair often incorporate leaflet materials with such reinforcement fibers for their load-bearing effects. It was hypothesized that important features of a closed, loaded valve can be controlled by varying global patterns of reinforcement fibers, and a finite element model of the aortic valve was used to study the effect of different fiber patterns on valve coaption and leaflet stress.

Materials and Methods

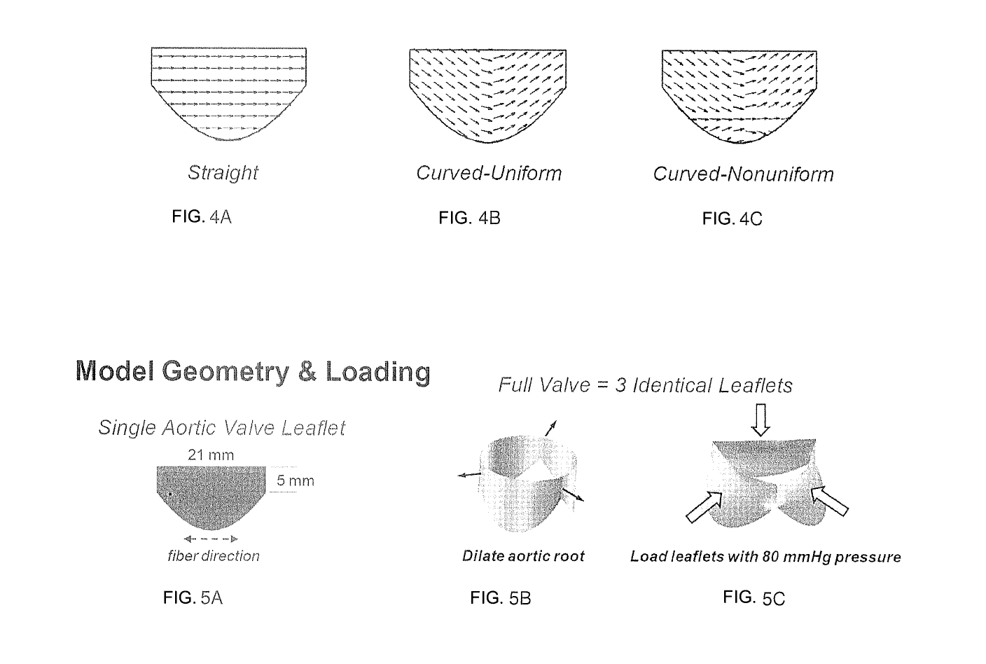

A dynamic finite element model of the aortic valve was used that incorporates a nonlinear anistropic constitutive law for the leaflet material. Three different leaflet fiber patterns were modeled: (1) a pattern of straight fibers parallel to the leaflet free edge (FIG. 4A), (2) a pattern of concave-up fibers opening toward the free edge (FIG. 4B), and (3) a spatially varying pattern with concave-up fibers in the top portion of the leaflet gradually changing to concave-down fibers near the bottom (FIG. 4C). The finite element model was used to simulate the state of the closed valve under end-diastolic pressure. A model of the geometry and loading for the valve leaflets is shown in FIGS. 5A-5C. The simulated closed state of the valve was assessed by computing the area of leaflet coaptation and the stresses in the leaflets.

Results and Discussion

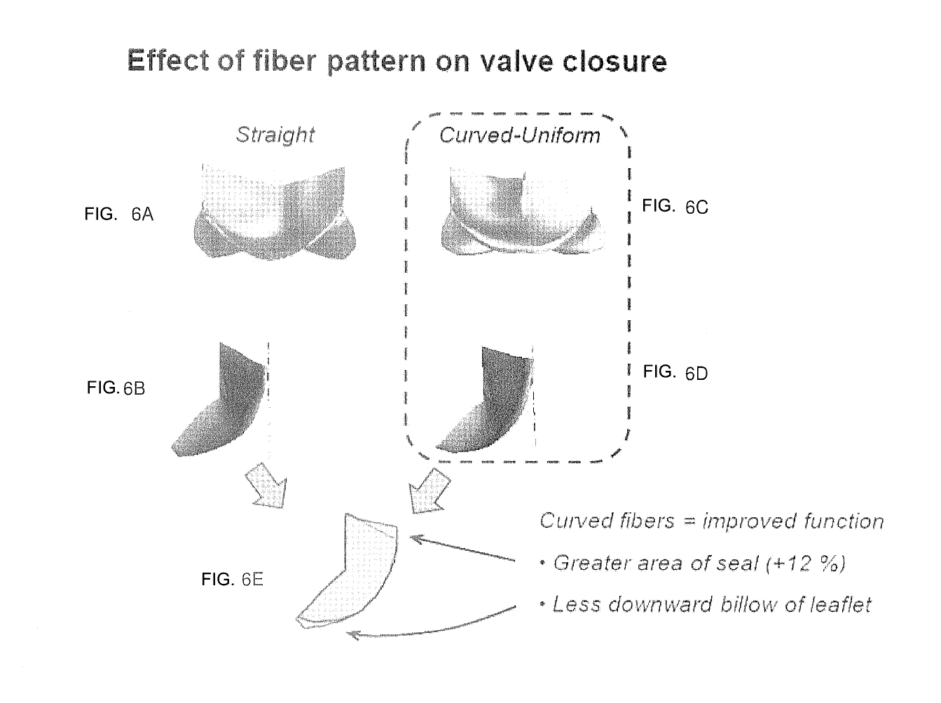

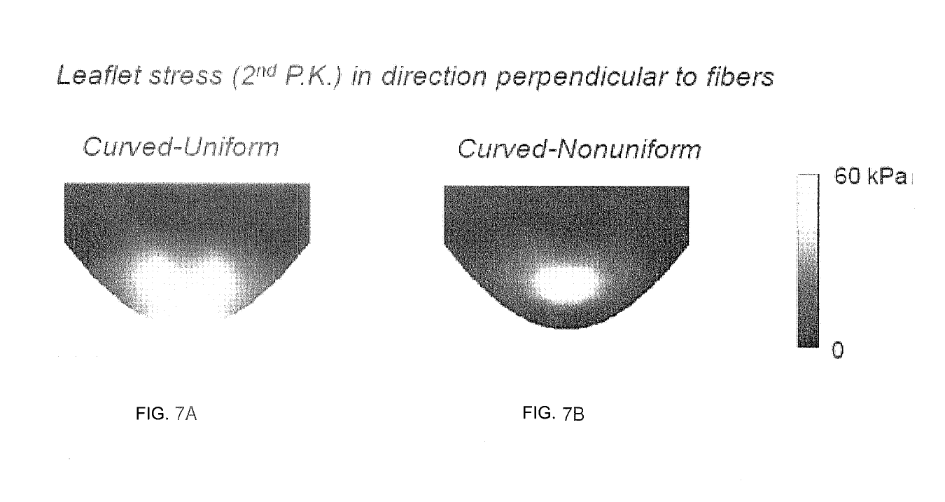

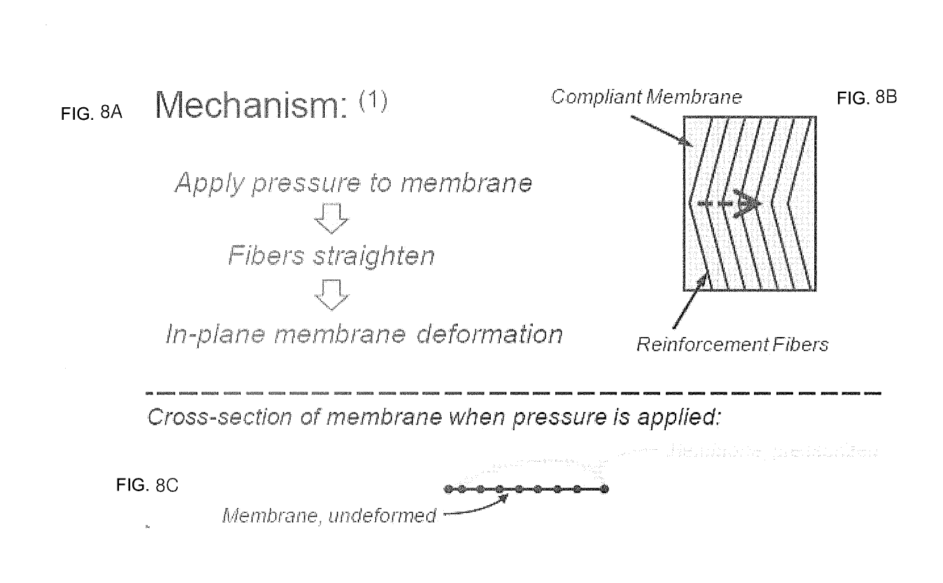

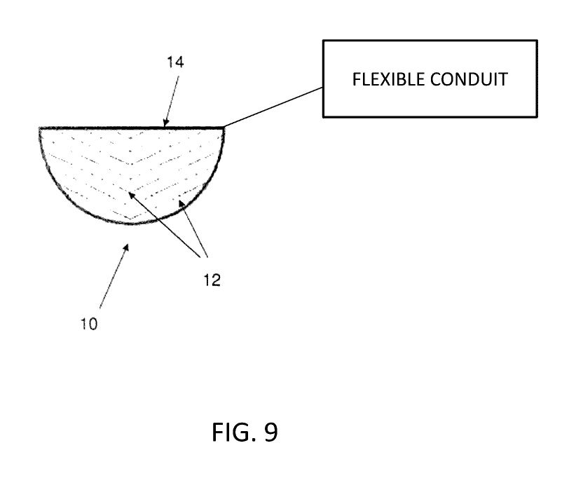

In the model with the concave-up pattern, the fibers tend to straighten as pressure loads the leaflets, causing in-plane deformation of the leaflet midline toward the free edge. This results in 12% greater area of leaflet coaptation than in the model with straight fibers as well as a flatter closed valve surface corresponding to more efficient valve function (as shown in FIGS. 6A-6E comparing the straight versus curved-uniform leaflet). However, it also introduces a stress concentration at the point of attachment of the bottom of the leaflet to the aortic root (FIG. 7A). In the model with the spatially varying pattern, the concave-up fiber pattern near the free edge increases the coaptation are by 13% compared to the model with straight fibers while the concave-down pattern near the bottom of the leaflet removes the stress concentration at the point of attachment, moving it toward the center of the leaflet where it can be counteracted by a local increase in leaflet thickness (FIG. 7B). The mechanism of the straightening of the leaflet fibers and deformation under pressure is summarized in FIGS. 8A-8C.

### CONCLUSIONS

Specific fiber patterns in heart valve leaflet material can be exploited to control the shape of the valve under pressure load and the stress field within the leaflets. This represents a potent and previously unreported mechanism that can be used in the design of prosthetic heart valves and in the design of leaflet grafts to be used in surgical repair of valves.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

1-24. (canceled)

25. A prosthetic heart valve, comprising three leaflets operatively attached together, wherein at least one leaflet includes a stretchable material and at least one of v-shaped or curved fibers, wherein the fibers are inextensible such that when the at least one leaflet is pressurized, a central portion of the fibers is straightened as the valve undergoes deformations and displacements tangent to a surface of the leaflet.

26. The prosthetic heart valve of claim 25, wherein the at least one leaflet is attached to a frame.

27. The prosthetic heart valve of claim 25, wherein the at least one leaflet is attached to a flexible conduit.

28. The prosthetic heart valve of claim 25, wherein said fibers are curved with respect to said free edge.

29. The prosthetic heart valve of claim 25, wherein said fibers are arranged in a V shape opening toward said free edge.

30. The prosthetic heart valve of claim 25, wherein said fibers are nonuniform and at least some of the fibers are arranged in a shape that opens toward the at least one free edge.

31. The prosthetic heart valve of claim 25, wherein the at least one leaflet is made of a plastic chosen from the group consisting of polytetrafluoroethylene, polyurethane, biaxially-oriented polyethylene terephthalate, and laminatable material.

32. The prosthetic heart valve of claim 25, wherein said fibers are made of a material chosen from the group consisting of carbon, polyester, aramid, and polyethylene.

33. The prosthetic heart valve of claim 25, wherein the at least one leaflet comprises a non-pressurized conformation and a pressurized conformation, wherein in the pressurized conformation, material of the at least one leaflet is pushed along a leaflet midline toward a free edge of the at least one leaflet.

34. The prosthetic heart valve of claim 25, wherein the valve comprises a non-pressurized conformation and a pressurized conformation, wherein, in the pressurized conformation, the at least one leaflet comprises a pocket arranged to increase coaptation height of the three leaflets, prevent billowing towards a ventricle and reduce stress in critical regions of the at least one leaflet.
